(19) United States
(12) Patent Application Publication
(10) Pub. No.: US 2013/0011876 A1
(43) Pub. Date: Jan. 10, 2013

(54) PROCESS FOR PREPARING FILAMENTOUS FUNGAL STRAINS HAVING A SEXUAL CYCLE AND A PROCESS FOR PREPARING SEXUALLY CROSSED FILAMENTOUS FUNGAL STRAINS

(75) Inventors: Peter Jozef Ida Van De Vondervoort, Schipluiden (NL); Herman Jan Pel, Terschuur (NL); Paul Stanley Dyer, Nottingham (GB)

(73) Assignee: DSM IP ASSETTS, B.V., Heerlen (NL)

(21) Appl. No.: 13/576,618

(22) PCT Filed: Jan. 12, 2011
(86) PCT No.: PCT/EP2011/050289

§ 371 Date: Sep. 7, 2012

(30) Foreign Application Priority Data
Feb. 4, 2010 (EP) 10152666.3

(51) Int. Cl. C12N 15/04; C12N 15/31; C07K 14/38; C40B 30/06; C12P 21/00; C12N 1/15; C12N 15/80

(52) U.S. Cl. 435/69.1; 435/454; 435/254.3; 536/23.74; 435/320.1; 506/10; 530/324

(57) ABSTRACT

The invention relates to a process for preparing filamentous fungal strains having a sexual cycle, wherein an acceptor filamentous fungal strain, having no or one type MAT locus, is subjected to recombination in which one or more mating type locus genes and optionally sex related genes, from other species than the acceptor filamentous fungal strain, is introduced into the acceptor filamentous fungal strain to produce a filamentous fungal strain having a sexual cycle.

### FIELD OF THE INVENTION

The invention relates to a process for preparing a filamentous fungal strain having a sexual cycle, and to the use thereof to sexually cross two individual strains resulting in new strains.

### BACKGROUND OF THE INVENTION

A process for preparing a filamentous fungal strain pair having a sexual cycle, is known from WO/2009/054330. WO/2009/054330 shows that replacement of the mating type locus of a Fusarium oxysporum with the opposite mating type locus of another isolate of the same species results in a pair of strains that are mating competent.

Pöggeler et al. 1997 (Mating type genes from the homothallic fungus Sordaria macrospora are functionally expressed in a heterothallic Ascomycete. Genetics 147:567-580) show that by transferring the homothallic mating locus of S. macrospora into Podospora ansinera, the latter can gain the ability to form fruiting bodies in a homothallic way.

Turgean et al 1993 (Cloning and analysis of the mating type genes from Cochliobolus heterostrophus. Mol. Gen. Genet. 238: 270-284) showed that by addition of one mating type locus MAT1-2 to a MAT1-1 containing strain, a functional homothallic strain was obtained.

Wirzel et al 1998 (Single mating type-specific genes and their 3′ UTRs control mating and fertility in Cochliobolus heterostrophus. Mol. Gen. Genet. 259: 272-281) show exchange of mating type loci altering the sexual identity in Cochliobolus heterosporus strains and their changed functionality in crosses.

Pöggeler et al. 2008 (Asexual Cephalosporin C producer Acremonium chrysogenum carries a functional mating type locus. Appl. Environm. Microbiol. 74: 6006-6016) show that the MAT1-1 locus of A. chysogenum can replace the MAT1-1 locus in Podospora ansinera for its function in a sexual cycle.

Grosse 2008 (The asexual pathogen Aspergillus fumigatus expresses functional determinants of Aspergillus nidulans sexual development Eukaryotic Cell, 7: 1724-1732) describes the functionality of the Aspergillus fumigatus MAT1-1 locus in the homothallic system of Aspergillus nidulans.

Kwon-Chung and Sugui 2009 (Sexual reproduction in Aspergillus species of medical or economical importance: why so fastidious. Trends in Microbiology 17:481-487) describe the recently discovered sexual ability of several Aspergillus species, previously thought to be restricted to asexual reproduction, and the differences in culture conditions required for performing the sexual cycle.

O'Gorman et al. 2009 (Discovery of a sexual cycle in the opportunistic fungal pathogen Aspergillus fumigatus, Nature 457, 22 Jan. 2009, 471-475) discloses pair wise crossing of MAT1-1 and MAT1-2 combinations of isolates of Aspergillus fumigatus.

The above prior art methods are however not useful for species for which only one mating type locus is known. For example, Aspergillus niger, cannot perform sexual crosses because only isolates with the MAT1-1 mating type locus are known so far. According to O'Gorman et al, cited above, for sexual development to occur in heterothallic fungi, it is necessary for the isolates to contain the opposite mating type loci. For example, for an Aspergillus niger strain with a MAT1-1 locus it would require an Aspergillus niger strain with the opposite MAT1-2 locus to have the two mating types present.

### SUMMARY OF THE INVENTION

It is therefore an object of the invention to provide a sexual cycle for filamentous fungal species for which no or only one mating type locus is found.

Another object of the invention is to provide a method for sexual crossing of different strains of a filamentous fungus species in which randomized exchange of chromosomal properties between the different strains can be recombined in a new individual. A further object is to provide such crossing method for strains based on a sexual cycle in which no selectable markers are needed.

Another object is to provide a process to sexually cross isogenic or near isogenic strains from one lineage of a species for which only one mating type locus is known, so that limited diversity can be introduced and exchanged without losing the advantageous properties common to the isogenic strains.

Another object is to provide a process to sexually cross the genetic backgrounds of two strains in a new strain.

One or more of these objects are attained according to the invention, that provides a process for the preparing filamentous fungal strain having a sexual cycle, wherein an acceptor filamentous fungal strain, having no or one type MAT locus, is subjected to recombination in which one or more mating type locus genes and optionally sex related genes, from other species from the same genus as the acceptor filamentous fungal strain, is introduced into the acceptor filamentous fungal strain to produce a filamentous fungal strain having a sexual cycle.

According to the invention, it is possible to obtain filamentous fungus individuals with an opposite mating type (e.g. the acceptor filamentous fungal strain and the strain resulting from the process) that have opposite mating type, resulting in one or more pair of strains with two opposite mating types and to strains having a functional sexual cycle.

According to the invention it further possible to construct a strain harbouring both mating type loci, that is able to reproduce homothallic or heterothallic, in a functional sexual cycle.

### BRIEF DESCRIPTION OF THE FIGURES

FIG. 1 depicts the strategy used to delete a gene or gene fragment from a host strain. The DNA construct used comprises the amdS selection marker flanked by homologous regions (5′ and 3′) of the gene to be deleted (1). This construct integrates through double homologous recombination (X) at the corresponding genomic locus (2) and replaces the genomic gene copy (3). Subsequently, recombination over the direct repeats (U) removes the amdS marker, resulting in precise excision of the gene or gene fragment to be deleted (4).

FIG. 2 shows in the top panel the genes in and surrounding the MAT1-2 locus of A. tubingensis isolate AN205 and in the bottom panel the orientation of genes in and surrounding the MAT1-1 locus of A. niger isolate CBS513.88. Gene numbers as annotated in the A. niger genome are given and homologous genes in the A. tubingens is genome are indicated by the same shading. The black bars indicate the sequences unique for the MAT-loci of the two isolates. In the top panel indicated by a dotted arrow the gene encoding the HMG-box containing protein and in the bottom panel indicated by an arrow with horizontal stripes is gene An11g10180 encoding the a-factor containing protein.

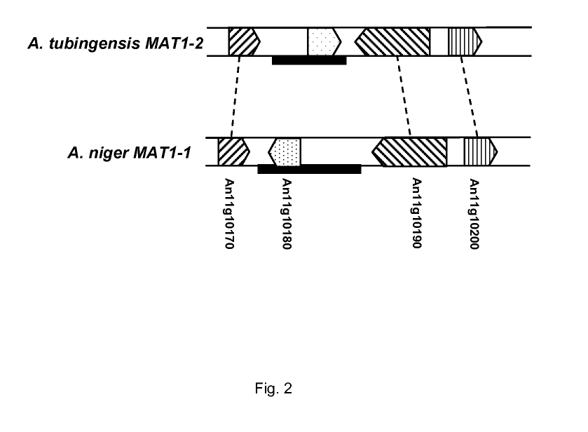

FIG. 3 is a schematic drawing of the MAT1-2 locus indicating the two PCR fragments generated by the primers listed in table 1 which are used in the construction of plasmid pGBmat1-2.

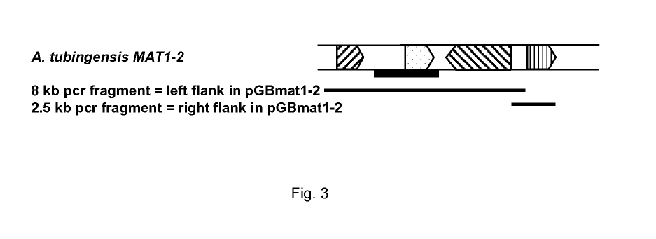

FIG. 4 Plasmid map of pGBMAT1-2 used to change the mating type of A. niger strains from MAT1-1 to MAT1-2.

FIG. 5 Schematic presentation of the replacement of the MAT1-1 locus of A. niger with the MAT1-2 locus of A. tubingensis. Panel 1 depicts the fragment plasmid pGBMAT1-2 containing the fungal DNA sequences. Panel 2 depicts the locus in the fungal strain that is subject for gene replacement, the lines connecting panel 1 and 2 indicating the areas where homologous recombination takes place. Panel 3 shows the A. niger genomic region after replacement of the “fragment to be replaced” by the “replacing fragment” including the selection marker amdS. Panel 4 shows the same locus after the amdS marker was removed by homologous recombination of the two identical flanks surrounding the amdS marker.

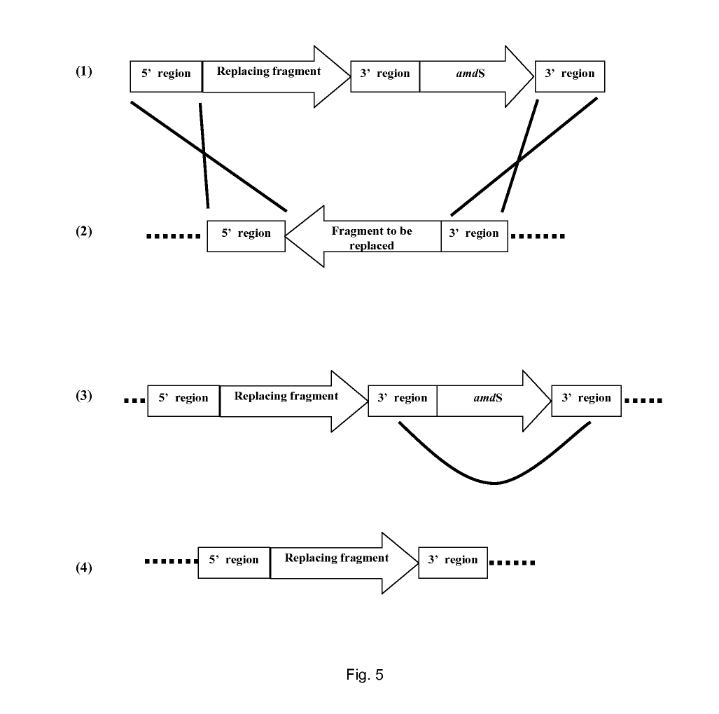

### BRIEF DESCRIPTION OF THE SEQUENCE LISTINGS

SEQ ID NO: 1 sets out the DNA sequence of the MAT1-2 locus of an Aspergillus tubingensis isolate.
SEQ ID NO: 2 sets out the amino acid sequence of the MAT1-2 locus protein of Aspergillus tubingensis.
SEQ ID NO: 3 sets out the amino acid sequence of the HMG-box from MAT1-2 of Aspergillus tubingensis.
SEQ ID NO: 4 sets out the amino acid sequence of the N-terminal part before the HMG box from MAT1-2 of Aspergillus tubingensis.
SEQ ID NO: 5 sets out the amino acid sequence of the MAT1-1 protein of Aspergillus niger. The code in Pel et al. 2007 under “Mating Processes”, “Signal Transduction—Sexual Reproduction” and “Ascomata (Fruit Body) Development, is An11g10180.

### DETAILED DESCRIPTION OF THE INVENTION

The acceptor filamentous fungal strain may be from any filamentous fungal species having no or one type MAT locus, these strains are unknown to have a sexual cycle so far. In one embodiment, the acceptor filamentous fungal strain has one or no or an unknown MAT locus. Filamentous fungal strains include, but are not limited to, strains of Acremonium, Agaricus, Aspergillus, Aureobasidium, Chrysosporium, Coprinus, Cryptococcus, Filibasidium, Fusarium, Humicola, Magnaporthe, Mucor, Myceliophthora, Neocallimastix, Neurospora, Paecilomyces, Penicillium, Piromyces, Panerochaete, Pleurotus, Schizophyllum, Talaromyces, Thermoascus, Thielavia, Tolypocladium, and Trichoderma.

Examples of suitable acceptor filamentous fungi having no sexual cycle and having no or one type MAT locus are Aspergillus niger, Aspergillus niger var. awamori, Aspergillus terreus, Aspergillus aculeatus, Aspergillus candidus, Aspergillus japonicus, Aspergillus sojae, Aspergillus vadensis, Aspergillus brasiliensis, Aspergillus carbonarius, Aspergillus lacticoffeatus, Aspergillus aureus, Aspergillus oryzae and Chrysosporium lucknowense.

In one embodiment, the acceptor filamentous fungus is of the genus Aspergillus. In an embodiment, the acceptor filamentous fungus is an Aspergillus species, such as Aspergillus niger, Aspergillus niger var. awamori, Aspergillus terreus, Aspergillus aculeatus, Aspergillus candidus, Aspergillus japonicus, or Aspergillus oryzae.

The one or more mating type locus genes and optionally one or more sex related genes, preferably not from the acceptor fungus species may originate from one or more donor fungus such as Aspergillus fischerianus, Aspergillus fumigatus, Aspergillus lentulus, Aspergillus tubingensis, Aspergillus fruticulosus, Aspergillus flavus, Aspergillus parasiticus, Aspergillus ochraceus, Aspergillus nidulans, Aspergillus flavipes, Aspergillus niveus, Aspergillus tetrazonus, or Aspergillus unguis or Renispora flavissima, or may be obtained otherwise, e.g. from another donor organism or by synthesis of the genes.

The donor fungus is preferably of another species from the same genus as the acceptor fungus. In one embodiment, of the donor strain at least two different isolates are available, one harbouring the MAT1-1 mating type determining locus the other harbouring the MAT1-2 mating type determining locus. In another embodiment, MAT1-1 and MAT1-2 are present in one donor strain. In one embodiment, the donor filamentous fungus is of genus Aspergillus. In an embodiment, the donor filamentous fungus is Aspergillus tubingensis. In another embodiment, the donor fungus is Aspergillus tubingensis AN205.

In an embodiment, the acceptor filamentous fungal strain is subjected to recombination with an isolated polynucleotide which comprises:
(a) the nucleotide sequence set out in SEQ ID NO: 1; or
(b) a nucleotide sequence having at least 70% sequence identity with the nucleotide sequence of SEQ ID NO: 1; or
(c) a sequence which is degenerate as a result of the genetic code to a sequence as defined in any one of (a), (b) or (d); or
(d) a nucleotide sequence which is the reverse complement of a nucleotide sequence as defined in (a), (b), or (c), or
(e) a nucleotide sequence encoding a polypeptide comprising the amino acid sequence set out in SEQ ID NO: 3 or a polypeptide having at least 70% sequence identity with the one of the sequence SEQ ID NO: 3, or
(f) a nucleotide sequence encoding a polypeptide comprising the amino acid sequence set out in SEQ ID NO: 4 or a polypeptide having at least 70% sequence identity with the one of the sequence SEQ ID NO: 4.

In an embodiment, the donor fungus and acceptor fungus are of the same species, but are different with respect to their MAT-loci and/or genes encoding putative mating type idiomorphs and/or sex-genes; e.g. the donor fungus may have one type of MAT locus and the acceptor fungus (having no sexual cycle) may have another type of MAT locus, wherein the donor MAT locus of the donor is able to complement the MAT locus of the acceptor fungus in order to confer a sexual cycle to the acceptor fungus.

Turgeon and Yoder 2000 (Proposed nomenclature for mating type genes of filamentous ascomycetes. Fungal Genetics and Biology 31:1-5) propose nomenclature for mating type genes, the definitions of which are also used herein.

According to the invention the mating type loci may be added to the acceptor, filamentous fungal strain or replace the existing mating type locus in the acceptor filamentous fungal strain. In one embodiment, when a mating type locus is added mating type locus MAT1-1 may be added to MAT1-2 or vice versa or MAT1-1 and MAT1-2 may be added to a strain having no mating type or presence of a mating type is unknown. In another MAT1-1 may be replaced by MAT1-2 or MAT1-2 may be replaced by MAT1-1. In one embodiment the pair consists of MAT1-1 and MAT1-2 or equally MAT1-2 and MAT1-1.

The resulting strains may be sexually crossed, where at least one MAT1-1 and one MAT1-2 form a pair. The following combinations are possible: MAT1-1×MAT1-2; MAT1-2×MAT1-1; MAT1-2+MAT1-1×MAT1-2; MAT1-1+MAT1-2×MAT1-1; MAT1-2+MAT1-1×MAT1-2+MAT1-1.

Accordingly in an embodiment of the invention the acceptor strain comprises MAT1-1 and from the donor strain MAT1-2 is introduced. In one embodiment thereof, both MAT1-1 and MAT1-2 may be present in the resulting strain and a homothallic sexual cycle may thus be introduced. Alternatively MAT1-1 may be replaced by MAT1-2, or MAT1-2 is introduced and MAT1-1 removed, resulting in a pair of strains (here the acceptor strain and the strain resulting from the recombination) and a heterothallic cycles may thus be introduced.

In another embodiment of the invention, the acceptor strain comprises no MAT, and from the donor strain both MAT1-1 and MAT1-2 is introduced. In one embodiment thereof, both MAT1-1 and MAT1-2 may be present in the resulting strain and a homothallic sexual cycle may thus be introduced. Alternatively in one recombination event MAT1-1 may be introduced into the acceptor strain, resulting in a strain with MAT1-1 mating type. In another recombination event, MAT1-2 may be introduced into the acceptor strain, resulting in a strain with MAT1-2 mating type. The strains of both recombination events then result in a pair of strains and a heterothallic cycle may thus be introduced.

In the process of the invention, the acceptor filamentous fungus is subjected to recombination in which one or more mating type locus genes and/or one or more sex related genes, from another species than the acceptor filamentous fungal species, e.g. originating from the donor fungus are introduced.

In the context of the present invention, the terms “recombination” and “recombinant” refers to any genetic modification not exclusively involving naturally occurring processes and/or genetic modifications induced by subjecting the host cell to random mutagenesis but also gene disruptions and/or deletions and/or specific mutagenesis, for example. Consequently, combinations of recombinant and naturally occurring processes and/or genetic modifications induced by subjecting the host cell to random mutagenesis are construed as being recombinant.

Recombination includes introduction and/or replacement of genes and may be executed by the skilled person using molecular biology techniques known to the skilled person (see: Sambrook & Russell, Molecular Cloning: A Laboratory Manual, 3rd Ed., CSHL Press, Cold Spring Harbor, N.Y., 2001). Examples of the general design of expression vectors for gene over expression and disruption vectors for down-regulation, transformation, use of markers and selective media can be found in WO199846772, WO199932617, WO2001121779, WO2005095624, EP 635574B and WO2005100573.

Genetic elements such as the mat-loci and/or genes encoding putative mating type idiomorphs and the sex-genes may e.g. be cloned and/or sequenced using different approaches, e.g.:

1) Using homology to closely related species of which genome sequence is known by designing primers preferably in coding regions of genes flanking the genetic elements of interest.

2) Full genome sequencing, followed by identification of the genes of interest by using bioinformatics can be done to identify the DNA sequence of genes involved

3) Genomic library construction and hybridization

4) Metagenomic library construction and sequencing and bioinformatics identification

5) Metagenomic library construction and high throughput screening for a (heterotallic or homothallic) functional sexual cycle of pairs of strains.

Repairing the Sexual Cycle:

In a preferred embodiment, the acceptor strain is adapted to obtain a strain with a locus encoding the mating type idiomorph. In another embodiment an acceptor strain is adapted to obtain a mating type MAT1-1 harbouring strain and a mating type MAT1-2 harbouring strain, both in which possibly defect genes required for a normal progression through the sexual cycle are repaired.

Sex-related genes are herein understood to be genes required for sexual reproduction of the fungus. In one embodiment, the sex-related genes are genes involved in mating behaviour, fertilization, spermatogenesis, or sex determination. In one embodiment, these functions may be performed by proteins that are expressed by the sex-related genes. Examples of such genes are listed in supplementary table 5 of Pel et al 2007 under “Mating Processes”, “Signal Transduction—Sexual Reproduction” and “Ascomata (Fruit Body) Development”, herein shown as table 1.

TABLE 1
Genes implicated in asexual and sexual reproduction in A. niger (from Pel et al, 2007)

| | | | | | |
|---|---|---|---|---|---|
| Gene¹ | Function | A. niger | A. nidulans | A. fumigatus | A. oryzae |

Mating Processes

| | | | | | |
|---|---|---|---|---|---|
| MAT-1 | (MAT-alpha1) Mating-type (alpha-box domain transcriptional | An11g10180 | AN2755.2 | Not found | 20164.m00251 |
| | activator) | | | | |
| MAT-2 | Mating-type (HMG-box transcriptional activator) | Not found | AN4734.2 | 59.m09249 | Not found |
| ppgA | (MFalpha 1 & 2) Pheromone precursor (alpha-factor like) | An18g06770 | AN5791.2 | 69.m14805 | 20086.m00080 |
| ppgB | (MFa1, MFa2) Pheromone precursor (a-factor like) | Ambiguous | Ambiguous | Not found | Not found |
| (KEX1) | Carboxypeptidase alpha-factor processing | An08g00430 | AN1384.2 | 70.m14837 | 20080.m00038 |
| kexB | (KEX2) Endoprotease for alpha-factor processing | An01g08530 | AN3583.2 | 58.m07381 | 20179.m00626 |
| (STE13) | Dipeptidyl aminopeptidase for alpha-factor processing | An02g11420 | AN2946.2 | 59.m09093 | 20174.m00466 |
| (STE23) | Dipeptidyl aminopeptidase for a-factor processing | An16g01860 | AN8044.2 | 53.m03900 | 20129.m00210 |
| (RCE1) | CAAX prenyl protease a-factor C-terminal processing | An14g03420 | AN6528.2 | 62.m03116 | 20107.m00091 |
| (STE24) | CAAX prenyl protease a-factor C and N-terminal processing | An04g01950 | Yes³ | 58.m07859 | 20142.m00264 |
| (RAM1/STE16) | CAAX-farnesyltransferase beta subunit; a-factor modification | An04g06620 | AN2002.2 | 58.m07610 | 20138.m00208 |
| (RAM2) | CAAX-farnesyltransferase alpha subunit; a-factor modification | An04g02210 | AN3867.2 | 58.m07839 | 20142.m00239 |
| (STE14) | CAAX-prenyl cysteine carboxymethyltransferase; a-factor | An12g03660 | AN6162.2 | 72.m19009 | 20177.m00422 |
| | modification | | | | |
| atrD (STE6) | ATP-dependent efflux pump for a-factor like pheromone | An04g03690 | AN2300.2 | 58.m08958 | 20136.m00137 |

Signal Transduction - Sexual Reproduction

| | | | | | |
|---|---|---|---|---|---|
| preB/gprA (STE 2) | Pheromone Receptor (for alpha-factor like pheromone) | An09g04180 | AN2520.2 | 59.m08468 | 20123.m00180 |
| preA/gprB (STE 3) | Pheromone Receptor (for a-factor like pheromone) | An03g03890 | AN7743.2 | 71.m15771 | 20162.m00356 |
| gprD | Receptor prevents improper sexual development | An02g01560 | AN3387.2 | 72.m19372 | 20141.m00118 |
| fadA (GPA1) | Alpha-subunit G protein | An08g06130 | AN0651.2 | 70.m15256 | 20180.m01174 |
| sfaD (STE 4) | Beta-subunit G protein | An18g02090 | AN0081.2 | 71.m15359 | 20148.m00279 |
| gpgA (STE18) | Gamma-subunit G protein | Partial | AN2742.2 | 54.m06689 | 20175.m00541 |
| | | sequence^{2,3} | | | |
| (STE20) | Serine/threonine protein kinase MKKKK | An11g04320 | AN2067.2 | 57.m05766 | 20178.m00629 |
| steC (STE11) | Serine/threonine protein kinase MKKK | An17g01280 | AN2269.2 | 71.m15914 | 20132.m00123 |
| STE7 | Serine/threonine protein kinase MKK | An11g10690 | AN3422.2 | 59.m09275 | 20164.m00222 |
| mpkB (FUS3) | Mitogen-activated protein kinase MK | An08g10670 | AN3719.2 | 69.m15727 | 20178.m00764 |
| steA (STE12) | Transcriptional Activator. Homeodomain DNA binding | An17g01580 | AN2290.2 | 71.m15938 | 20132.m00151 |
| (STE50) | Pheromone adaptation feedback response | An04g09220 | AN7252.2 | 72.m19797 | 20134.m00115 |
| Nc ham-2 (FAR11) | Transmembrane protein required for mating cell fusion | An15g01470 | AN6611.2 | 62.m03182 | 20163.m00245 |
| fphA | Red light phytochrome | An14g02970 | AN9008.2 | 89.m01927 | 20173.m00405 |
| sakA | MAP kinase represses sexual development | An08g05850 | AN1017.2 | 70.m15235 | 20180.m01196 |

Ascomata (Fruit Body) Development

| | | | | | |
|---|---|---|---|---|---|
| veA | Velvet activator induces sexual reproduction A. nidulans | An08g05100 | AN1052.2 | 70.m15191 | 20173.m00346 |
| nsdD | GATA-transcription factor, light regulation | An02g09610 | AN3152.2 | 59.m08512 | 20010.m00003 |
| csnD | Signalosome subunit 4, regulation of sexual development | An16g07210 | AN1539.2 | 55.m03058 | 20171.m00527 |
| csnE | Signalosome subunit 5, regulation of sexual development | An15g06660 | AN2129.2 | 72.m19712 | 20115.m00100 |
| dopA | Leucine zipper-like domain regulator (initiation morphogenesis) | An11g04750 | AN2094.2 | 57.m05797 | 20178.m00666 |
| pro1 | Cys6-Zn2 transcriptional activator; maturation ascomata | An04g07400⁴ | AN1848.2 | 58.m07662 | 20129.m00151 |
| pro11 | WD40 scaffold protein; regulation fruit body formation | An16g01520 | AN8071.2 | 53.m03924 | 20167.m00272 |
| mutA | Mutanase, cell wall turnover during sexual development | An06g00510 | AN7349.2 | 57.m05694 | 20150.m00367 |
| stuA | APSES-transcription factor | An05g00480 | AN5836.2 | 72.m19916 | 20177.m00377 |
| medA | Transcription factor | An02g02150² | AN6230.2 | 72.m19977 | 20141.m00193 |
| Nc asd-1 | Ascus development; rhamnogalacuronase B | An14g01130 | AN7135.2 | 89.m02015 | 20153.m00219 |
| Nc asd-4/areB | Ascus development; GATA-Zn finger transcription factor | An02g02240 | AN6221.2 | 72.m19434 | 20141.m00163 |
| Pa cro1 (SHE4) | Regulator of myosin; required for syncytial to cellular transition | An18g03150 | AN0135.2 | 71.m15419 | 20158.m00313 |
| esdC | Required for sexual development in A. nidulans | An12g00710 | AN9121.2 | 66.m04577 | 20169.m00374 |
| ppoA | Fatty acid oxygenase for Psi factor production | An04g05880 | AN1967.2 | 58.m07572 | 20138.m00158 |
| ppoB | Fatty acid oxygenase for Psi factor production | No definite | AN6320.2 | No definite | 20156.m00214 |
| | | match | | match | |
| ppoC | Fatty acid oxygenase for Psi factor production | An02g07930 | AN5028.2 | 59.m09493 | 20175.m00495 |
| Gz ppoD | Fatty acid oxygenase | An12g01320 | No definite | No definite | 20140.m00151 |
| | | | match | match | |

¹Gene name derived from Aspergillus nidulans where known. Names in parentheses are from Saccharomyces cerevisiae. Prefix Nc = Neurospora crassa; Pa = Podospora anserina
²Partial Sequence found at end/start of contig.
³Present in genome but not detected by autoannotation.
⁴Contains internal stop codon.

The invention further relates to filamentous fungal strains having a sexual cycle, obtainable by the process of the invention as described above.

The invention further relates to a polynucleotide which comprises:

(a) the nucleotide sequence set out in SEQ ID NO: 1; or

(b) a nucleotide sequence having at least 70% sequence identity, at least 75% sequence identity, at least 80% sequence identity, at least 85% sequence identity, at least 90% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity with the nucleotide sequence of SEQ ID NO: 1; or

(c) a sequence which is degenerate as a result of the genetic code to a sequence as defined in any one of (a), (b), or (d); or

(d) a nucleotide sequence which is the reverse complement of a nucleotide sequence as defined in (a), (b), or (c).

The invention further relates to a polypeptide comprising the amino acid sequence set out in SEQ ID NO: 2 or a variant polypeptide thereof having at least 70% sequence identity, at least 75% sequence identity, at least 80% sequence identity, at least 85% sequence identity, at least 90% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity with the one of the sequences SEQ ID NO: 2.

The invention further relates to a polypeptide comprising the amino acid sequence set out in SEQ ID NO: 3 or a variant polypeptide thereof having at least 70% sequence identity, at least 75% sequence identity, at least 80% sequence identity, at least 85% sequence identity, at least 90% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity with the one of the sequences SEQ ID NO: 3.

The invention further relates to a polypeptide comprising the amino acid sequence set out in SEQ ID NO: 4 or a variant polypeptide thereof having at least 70% sequence identity, at least 75% sequence identity, at least 80% sequence identity, at least 85% sequence identity, at least 90% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity with the one of the sequences SEQ ID NO: 4.

Sequence identity is preferably calculated over the entire length of the polynucleotide molecule or polypeptide molecule.

The invention further relates to a vector incorporating the polynucleotide or any polynucleotide encoding one or more of the polypeptide(s) of the invention as defined above.

The polynucleotide of interest encoding a compound of interest, or DNA construct comprising the polynucleotide of interest and control sequences described above may be joined together to produce a recombinant expression vector which may include one or more convenient restriction sites to allow for insertion or substitution of optional polynucleotide sequences.

Alternatively, the polynucleotide of interest may be expressed by inserting the sequence or a nucleic acid construct comprising polynucleotide of interest into an appropriate vector for expression. In creating the expression vector, the coding sequence is located in the vector so that the coding sequence is operably linked with the appropriate control sequences for expression, and possibly secretion.

The recombinant expression vector may be any vector (e.g., a plasmid or virus), which can be conveniently subjected to recombinant DNA procedures and can bring about the expression of the polynucleotide of interest. The choice of the vector will typically depend on the compatibility of the vector with host cell into which the vector is to be introduced. The vectors may be linear or closed circular plasmids. The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. An autonomously maintained cloning vector may comprise the AMA1-sequence (see e.g. Aleksenko and Clutterbuck (1997), Fungal Genet. Biol. 21: 373-397).

Alternatively, the polynucleotide of interest may be expressed by inserting the sequence or a nucleic acid construct comprising polynucleotide of interest into an appropriate vector for expression. In creating the expression vector, the coding sequence is located in the vector so that the coding sequence is operably linked with the appropriate control sequences for expression, and possibly secretion.

The recombinant expression vector may be any vector (e.g., a plasmid or virus), which can be conveniently subjected to recombinant DNA procedures and can bring about the expression of the polynucleotide of interest. The choice of the vector will typically depend on the compatibility of the vector with host cell into which the vector is to be introduced. The vectors may be linear or closed circular plasmids. The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. An autonomously maintained cloning vector may comprise the AMA1-sequence (see e.g. Aleksenko and Clutterbuck (1997), Fungal Genet. Biol. 21: 373-397).

Alternatively, the vector may be one which, when introduced into the host cell, is integrated into the genome and replicated together with the chromosome (s) into which it has been integrated. Preferably, the integrative cloning vector comprises a DNA fragment, which is homologous to a DNA sequence in a predetermined target locus in the genome of the host cell for targeting the integration of the cloning vector to this predetermined locus. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least 30 bp, preferably at least 50 bp, preferably at least 0.1 kb, even preferably at least 0.2 kb, more preferably at least 0.5 kb, even more preferably at least 1 kb, most preferably at least 2 kb.

The vector system may be a single vector or plasmid or two or more vectors or plasmids, which together contain the total DNA to be introduced into the genome of the filamentous fungal cell, or a transposon.

The vectors preferably contain one or more selectable markers, which permit easy selection of transformed cells. A selectable marker is a gene the product of which provides for biocide or viral resistance, resistance to heavy metals, prototrophy to auxotrophs, and the like. A selectable marker for use in a filamentous fungal host cell may be selected from the group including, but not limited to, amdS (acetamidase), argB (ornithine carbamoyltransferase), bar (phosphinothricinacetyltransferase), b/eA (phleomycin binding), hygB (hygromycinphosphotransferase), niaD (nitrate reductase), pyrG (orotidine-5′-phosphate decarboxylase), sC (sulfate adenyltransferase), and trpC (anthranilate synthase), as well as equivalents from other species. Preferred for use in an Aspergillus and Penicillium cell are the amdS (EP 635574 B1, WO 97/06261) and pyrG genes of A. nidulans or A. oryzae and the bar gene of Streptomyces hygroscopicus. More preferably an amdS gene is used, even more preferably an amdS gene from A. nidulans or A. niger. A most preferred selection marker gene is the A. nidulans amdS coding sequence fused to the A. nidulans gpdA promoter (see EP 635574 B1). AmdS genes from other filamentous fungi may also be used (WO 97/06261).

The invention further relates to a filamentous fungal cell comprising a polynucleotide of the invention as defined above, a polypeptide of the invention as defined above and/or a vector of the invention as defined above.

In an embodiment, the invention relates to a process for producing filamentous fungal strain progeny, wherein individuals with two opposite mating types of the filamentous of the invention as defined above are crossed and progeny is isolated.

In an embodiment of the invention the process for selecting a filamentous fungal strain phenotype wherein a library of progeny of fungus with sexual cycle according to the invention as defined above is produced and is screened and a strain with particular phenotype is selected.

In an embodiment, in the process for selecting a filamentous fungal strain phenotype, wherein two opposite mating type filamentous fungal strains from a pool of strains, that are of vertical or horizontal lineage, are sexually crossed. In this way, strains may be obtained that are nearly isogenic and limited diversity can be introduced, without losing the advantageous properties common to strains in the highly isogenic pool of strains.

The MAT1-1 and MAT1-2 mating type genes of Fusarium oxysporum are less than 50% identical to the mating type genes of Aspergilli.

In one embodiment, the restoring of the sexual cycle, in a species of which no sexual cycle is observed, occurs by using genetic elements or genes from a closely related species with an intact sexual cycle.

This invention helps to recombine properties of two strains of the same species in an effective way, i.e. by sexual crossing. The advantage of the current invention to the parasexual cycle is that mutations from different strains can be recombined in progeny, that randomized exchange of chromosomal properties between the different strains can be recombined. Further, no markers are needed for the crossing process.

The properties that may be favourably be introduced by the exchange include but are not limited to additional properties (insertion), deleted properties (deletion) or alterated properties (mutation). Examples are for instance deletion of toxin clusters, protease deletions, regulator deletions, and production of compounds or interest. Examples of properties are described in more detail hereafter.

A number of host cells, especially fungi, which are used as host cells in the production of polypeptides of interest possesses genes encoding enzymes involved in the biosynthesis of various toxins. For example, cyclopiazonic acid, kojic acid, 3-nitropropionic acid and aflatoxins are known toxins, which are formed in, e.g., Aspergillus flavus. Similarly, trichothecenes are formed in a number of fungi, e.g., in Fusarium sp. such as Fusarium venenatum and in Trichoderma and ochratoxin may be produced by Aspergillus. Recently, sequencing of the genome of an industrial Aspergillus niger host strain revealed a fumonisin gene cluster (Pel et al., “Genome sequencing and analysis of the versatile cell factory Aspergillus niger CBS 513.88”. Nat. Biotechnol. 2007 February; 25 (2):221-231). The formation of such toxins during the fermentation of compounds of interest is highly undesirable as these toxins may present a health hazard to operators, customers and the environment. Consequently, a toxin deficient host cell enables toxin-free production of a compound of interest. The toxin-free compound is easier to produce since no toxin has to be removed from the product. Furthermore, the regulatory approval procedure for the compound is easier.

In an embodiment, a strain according to the invention may comprise a toxin associated polynucleotide encoding a compound (which may e.g. be a polypeptide which which may be an enzyme) or biochemical pathway, said toxin associated polynucleotide comprising a modification, wherein the host cell is deficient in the production of said toxin or a toxin intermediate compound compared to the parent cell it originates from when cultivated under comparable conditions. Preferably, the toxin or toxin intermediate compound is a fungal toxin or toxin intermediate compound. More preferably, the toxin or toxin intermediate compound is a toxin or toxin intermediate compound from Aspergillus. Even more preferably the toxin or the toxin intermediate compound is a toxin or toxin intermediate compound from Aspergillus niger. Even more preferably the toxin or toxin intermediate compound is a toxin or toxin intermediate compound from Aspergillus niger CBS 513.88. Even more preferably, the toxin or the toxin intermediate compound is fumonisin or a fumonisin intermediate compound. Even more preferably, the toxin or the toxin intermediate compound is ochratoxin or an ochratoxin intermediate compound. Most preferably, the toxin or the toxin intermediate compound is ochratoxin or fumonisin or an ochratoxin or a fumonisin intermediate compound.

The toxin associated polynucleotide encodes a compound (which may e.g. be a polypeptide which may be an enzyme) or a biochemical pathway which is involved in the production of a fungal toxin or toxin intermediate compound. More preferably, a toxin or toxin intermediate compound from Aspergillus. Even more preferably, a toxin or toxin intermediate compound from Aspergillus niger. Even more preferably, a toxin or toxin intermediate compound from Aspergillus niger CBS 513.88. Even more preferably, a fumonisin or a fumonisin intermediate compound. Even more preferably, a fumonisin-B or a fumonisin-B intermediate compound. Even more preferably, the toxin associated polynucleotide comprises the sequence of the fumonisin cluster from An01g06820 until An01g06930. Most preferably, the toxin associated polynucleotide comprises the sequence of An01g06930.

In another embodiment, the toxin associated polynucleotide encodes a compound (which may e.g. be a polypeptide which may be an enzyme) or a biochemical pathway which is involved in ochratoxin or an ochratoxin intermediate compound. More preferably, the toxin associated polynucleotide comprises the sequence of the cluster from An15g07880 until An15g07930. Most preferably, the toxin associated polynucleotide comprises the sequence of An15g07910. Most preferably, the toxin associated polynucleotide comprises the sequence of An15g07920.

Preferably, the host cell according to the invention comprises at least one toxin associated polynucleotide encoding a compound (which may e.g. be a polypeptide which may be an enzyme) or biochemical pathway, said toxin associated polynucleotide comprising at least one modification, wherein the host cell is deficient in the production of a toxin or, toxin intermediate compound compared to the parent cell it originates from when cultivated under comparable conditions.

More preferably, the host cell according to the invention comprises two toxin associated polynucleotides, said two toxin associated polynucleotides each comprising at least one modification, wherein the host cell is preferably deficient in the production of fumonisin and ochratoxin compared to the parent cell it originates from when cultivated under comparable conditions.

Even more preferably, the host cell according to the invention comprises three or more toxin associated polynucleotides said three or more toxin associated polynucleotides each comprising at least one modification, wherein the host cell is preferably deficient in the production of fumonisin, ochratoxin and at least one additional toxin or toxin intermediate compound compared to the parent cell it originates from when cultivated under comparable conditions.

In an embodiment, the strain according to the invention is deficient in glaA and at least one of the components selected from the group of amyA, amyBI, amyBII, oahA, toxin associated compound and prtT, by virtue of having a modification in the polynucleotide encoding glaA and said components. More preferably, the host cell according to the invention is deficient in glaA, oahA and at least one of the components selected from the group of amyA, amyBI, amyBII, toxin associated compound and prtT Even more preferably, the host cell according to the invention is deficient in glaA, oahA, toxin associated compound and at least one of the components selected from the group of amyA, amyBI, amyBII, and prtT. Even more preferably the host cell according to the invention is deficient in glaA, oahA, amyA, amyBI, amyBII and at least one of the components selected from the group of toxin associated compound and prtT. Even more preferably the host cell according to the invention is deficient in glaA, oahA, amyA, amyBI, amyBII, prtT and toxin associated compound. Most preferably, the host cell according to the invention comprising at least two substantially homologous DNA domains suitable for integration of one or more copies of a polynucleotide of interest wherein at least one of the at least two substantially homologous DNA domains is adapted to have enhanced integration preference for the polynucleotide of interest compared to the substantially homologous DNA domain it originates from, is deficient in glaA, oahA, amyA, amyBI, amyBII, prtT and toxin associated compound and is furthermore deficient in an NHR component, preferably ku70 or a homologue thereof.

In addition to deficiency or modification of the components described here above, the host cell according to the invention may be deficient in other components, such as major proteases like pepA. Preferably, the pepA is a fungal pepA. More preferably, the pepA is the pepA from Aspergillus. Even more preferably the pepA is the pepA from Aspergillus niger. Even more preferably the pepA is the pepA from Aspergillus niger CBS 513.88. Most preferably, the pepA comprises the sequence of An14g04710. Preferably, the host cell according to the invention demonstrates at least 5% deficiency of pepA, more preferably at least 10% deficiency, more preferably at least 20% deficiency, more preferably at least 30% deficiency, more preferably at least 40% deficiency, more preferably at least 50% deficiency, more preferably at least 60% deficiency, more preferably at least 70% deficiency, more preferably at least 80% deficiency, more preferably at least 90% deficiency, more preferably at least 95% deficiency, more preferably at least 97% deficiency, more preferably at least 99% deficiency. Most preferably, the host cell demonstrates 100% deficiency of pepA.

Alternatively, or in combination with the deficiencies described here above, the strain according to the invention may comprise an elevated unfolded protein response (UPR) compared to the wild type cell to enhance production abilities of a polypeptide of interest. UPR may be increased by techniques described in US2004/0186070A1 and/or US2001/0034045A1 and/or WO01/72783A2 and/or WO2005/123763. More specifically, the protein level of HAC1 and/or IRE1 and/or PTC2 has been modulated, and/or the SEC61 protein has been engineered in order to obtain a host cell having an elevated UPR. A preferred SEC61 modification is a modification which results in a one-way mutant of SEC61; i.e. a mutant wherein the de novo synthesized protein can enter the ER via SEC61, but the protein cannot leave the ER via SEC61. Such modifications are extensively described in WO2005/123763. Most preferably, the SEC 61 modification is the S376W mutation in which Serine 376 is replaced by Tryptophan.

The present invention also provides for a method for the production of a compound of interest comprising
a. cultivating a crossed cell according to the invention under conditions conducive to the production of said compound; and
b. recovering the compound of interest from the cultivation medium.

The compound of interest of the present invention can be any biological compound. The biological compound may be any biopolymer or metabolite. The biological compound may be encoded by a single polynucleotide or a series of polynucleotides composing a biosynthetic or metabolic pathway or may be the direct result of the product of a single polynucleotide or products of a series of polynucleotides. The biological compound may be native to the host cell or heterologous.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. Polypeptides further include naturally occurring allelic and engineered variations of the above-mentioned polypeptides and hybrid polypeptides. The polypeptide may native or may be heterologous to the host cell. The polypeptide may be a collagen or gelatin, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, polyphenoloxidase, ribonuclease, transglutaminase, or glucose oxidase, hexose oxidase, monooxygenase.

In the methods of the present invention, a polypeptide can also be a fused or hybrid polypeptide to which another polypeptide is fused at the N-terminus or the C-terminus of the polypeptide or fragment thereof. A fused polypeptide is produced by fusing a nucleic acid sequence (or a portion thereof) encoding one polypeptide to a nucleic acid sequence (or a portion thereof) encoding another polypeptide.

Techniques for producing fusion polypeptides are known in the art, and include, ligating the coding sequences encoding the polypeptides so that they are in frame and expression of the fused polypeptide is under control of the same promoter (s) and terminator. The hybrid polypeptides may comprise a combination of partial or complete polypeptide sequences obtained from at least two different polypeptides wherein one or more may be heterologous to the host cell.

The biopolymer may be a polysaccharide. The polysaccharide may be any polysaccharide, including, but not limited to, a mucopolysaccharide (e.g., heparin and hyaluronic acid) and nitrogen-containing polysaccharide (eg., chitin). In a more preferred option, the polysaccharide is hyaluronic acid.

The polynucleotide of interest according to the invention may encode an enzyme involved in the synthesis of a primary or secondary metabolite, such as organic acids, carotenoids, (beta-lactam) antibiotics, and vitamins. Such metabolite may be considered as a biological compound according to the present invention.

The term “metabolite” encompasses both primary and secondary metabolites; the metabolite may be any metabolite. Preferred metabolites are citric acid, gluconic acid and succinic acid.

The metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

The primary metabolite may be, but is not limited to, an amino acid, fatty acid, nucleoside, nucleotide, sugar, triglyceride, or vitamin.

The secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams.

The biological compound may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitratereductase), pyrG (orotidine-5′-phosphate decarboxylase), sC (sulfate adenyltransferase), trpC (anthranilate synthase), ble (phleomycin resistance protein), as well as equivalents thereof.

According to the invention, the compound of interest in the methods according to the invention is preferably a polypeptide as described herein.

Preferably, the polypeptide in the methods according to the invention is an enzyme as described herein.

According to the invention, the compound of interest in the methods according to the invention is preferably a metabolite.

In the production methods according to the present invention, the filamentous fungal strains according to the invention are cultivated in a nutrient medium suitable for production of the compound of interest, e.g. polypeptide or metabolite using methods known in the art. Examples of cultivation methods which are not construed to be limitations of the invention are submerged fermentation, surface fermentation on solid state and surface fermentation on liquid substrate. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the coding sequence to be expressed and/or the polypeptide to be isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide or metabolite is secreted into the nutrient medium, the polypeptide or metabolite can be recovered directly from the medium. If the polypeptide or metabolite is not secreted, it can be recovered from cell lysates.

Polypeptides may be detected using methods known in the art that are specific for the polypeptides. These detection methods may include use of specific antibodies, formation of an enzyme product, or disappearance of an enzyme substrate.

The resulting compound of interest e.g. polypeptide or metabolite may be recovered by the methods known in the art. For example, the polypeptide or metabolite may be recovered from the nutrient medium by conventional procedures including, but not limited to, centrifugation, filtration, extraction, spray-drying, evaporation, or precipitation.

Polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulphate precipitation), SDS-PAGE, or extraction (see, e.g., Protein Purification, J.-C. Janson and Lars Ryden, editors, VCH Publishers, New York, 1989).

Preferably, the cell in the process according to the invention is a selection marker free host cell as described earlier herein.

Preferably, the host cell in the methods according to the invention is a filamentous fungal host cell as described earlier herein.

The compound of interest of the present invention can be any biological compound. The biological compound may be any biopolymer or metabolite. The biological compound may be encoded by a single polynucleotide or a series of polynucleotides composing a biosynthetic or metabolic pathway or may be the direct result of the product of a single polynucleotide or products of a series of polynucleotides. The biological compound may be native to the host cell or heterologous.

The term “heterologous biological compound” is defined herein as a biological compound which is not native to the cell; or a native biological compound in which structural modifications have been made to alter the native biological compound.

The term “biopolymer” is defined herein as a chain (or polymer) of identical, similar, or dissimilar subunits (monomers). The biopolymer may be any biopolymer. The biopolymer may for example be, but is not limited to, a nucleic acid, polyamine, polyol, polypeptide (or polyamide), or polysaccharide.

The biopolymer may be a polypeptide. The polypeptide may be any polypeptide having a biological activity of interest. The term “polypeptide” is not meant herein to refer to a specific length of the encoded product and, therefore, encompasses peptides, oligopeptides, and proteins. Polypeptides further include naturally occurring allelic and engineered variations of the above-mentioned polypeptides and hybrid polypeptides. The polypeptide may native or may be heterologous to the host cell. The polypeptide may be a collagen or gelatin, or a variant or hybrid thereof. The polypeptide may be an antibody or parts thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase. The polypeptide may be an enzyme secreted extracellularly. Such enzymes may belong to the groups of oxidoreductase, transferase, hydrolase, lyase, isomerase, ligase, catalase, cellulase, chitinase, cutinase, deoxyribonuclease, dextranase, esterase. The enzyme may be a carbohydrase, e.g. cellulases such as endoglucanases, β-glucanases, cellobiohydrolases or β-glucosidases, hemicellulases or pectinolytic enzymes such as xylanases, xylosidases, mannanases, galactanases, galactosidases, pectin methyl esterases, pectin lyases, pectate lyases, endo polygalacturonases, exopolygalacturonases rhamnogalacturonases, arabanases, arabinofuranosidases, arabinoxylan hydrolases, galacturonases, lyases, or amylolytic enzymes; hydrolase, isomerase, or ligase, phosphatases such as phytases, esterases such as lipases, proteolytic enzymes, oxidoreductases such as oxidases, transferases, or isomerases. The enzyme may be a phytase. The enzyme may be an aminopeptidase, asparaginase, amylase, carbohydrase, carboxypeptidase, endo-protease, metallo-protease, serine-protease catalase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, protein deaminase, invertase, laccase, lipase, mannosidase, mutanase, oxidase, pectinolytic enzyme, peroxidase, phospholipase, polyphenoloxidase, ribonuclease, transglutaminase, or glucose oxidase, hexose oxidase, monooxygenase.

The biopolymer may be a polysaccharide. The polysaccharide may be any polysaccharide, including, but not limited to, a mucopolysaccharide (e.g., heparin and hyaluronic acid) and nitrogen-containing polysaccharide (e.g., chitin). In a more preferred option, the polysaccharide is hyaluronic acid.

The polynucleotide of interest according to the invention may encode an enzyme involved in the synthesis of a primary or secondary metabolite, such as organic acids, carotenoids, (beta-lactam) antibiotics, and vitamins. Such metabolite may be considered as a biological compound according to the present invention.

The term “metabolite” encompasses both primary and secondary metabolites; the metabolite may be any metabolite. Preferred metabolites are citric acid, gluconic acid and succinic acid.

The metabolite may be encoded by one or more genes, such as in a biosynthetic or metabolic pathway. Primary metabolites are products of primary or general metabolism of a cell, which are concerned with energy metabolism, growth, and structure. Secondary metabolites are products of secondary metabolism (see, for example, R. B. Herbert, The Biosynthesis of Secondary Metabolites, Chapman and Hall, New York, 1981).

The primary metabolite may be, but is not limited to, an amino acid, fatty acid, nucleoside, nucleotide, sugar, triglyceride, or vitamin.

The secondary metabolite may be, but is not limited to, an alkaloid, coumarin, flavonoid, polyketide, quinine, steroid, peptide, or terpene. The secondary metabolite may be an antibiotic, antifeedant, attractant, bacteriocide, fungicide, hormone, insecticide, or rodenticide. Preferred antibiotics are cephalosporins and beta-lactams.

The biological compound may also be the product of a selectable marker. A selectable marker is a product of a polynucleotide of interest which product provides for biocide or viral resistance, resistance to heavy metals, prototrophy to auxotrophs, and the like. Selectable markers include, but are not limited to, amdS (acetamidase), argB (ornithinecarbamoyltransferase), bar (phosphinothricinacetyltransferase), hygB (hygromycin phosphotransferase), niaD (nitratereductase), pyrG (orotidine-5′-phosphate decarboxylase), sC (sulfate adenyltransferase), trpC (anthranilate synthase), ble (phleomycin resistance protein), as well as equivalents thereof.

According to the invention, the compound of interest in the methods according to the invention is preferably a polypeptide as described herein.

Preferably, the polypeptide in the methods according to the invention is an enzyme as described herein.

According to the invention, the compound of interest in the methods according to the invention is preferably a metabolite.

The sequence information as provided herein should not be so narrowly construed as to require inclusion of erroneously identified bases. The specific sequences disclosed herein can be readily used to isolate the complete gene from the respective host cells which in turn can easily be subjected to further sequence analyses thereby identifying sequencing errors.

Unless otherwise indicated, all nucleotide sequences determined by sequencing a DNA molecule herein were determined using an automated DNA sequencer and all amino acid sequences of polypeptides encoded by DNA molecules determined herein were predicted by translation of a nucleic acid sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

The person skilled in the art is capable of identifying such erroneously identified bases and knows how to correct for such errors.

The invention described and claimed herein is not to be limited in scope by the specific embodiments herein enclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. In case of conflict, the present disclosure including definitions will be taken as a guide.

The invention will be illustrated below.

### EXAMPLES

### Example I

### Construction of Aspergillus niger Strains Having a Heterothallic Sexual Cycle

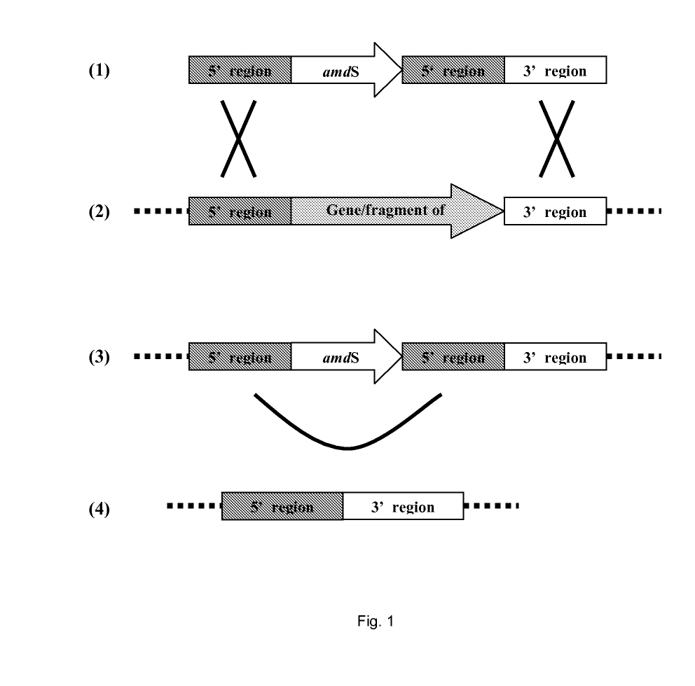

All gene replacement vectors described and used below, were designed according to known principles and constructed according to routine cloning procedures. In essence, these vectors comprise approximately 1-2 kb flanking regions of the respective ORF sequences, to target for homologous recombination at the predestined genomic loci. In addition, they contain the A. nidulans bi-directional amdS selection marker for transformation, in-between direct repeats. The method applied for gene deletion in all examples herein uses linear DNA, which integrates into the genome at the homologous locus of the flanking sequences by a double cross-over, thus substituting the gene to be deleted by the amdS gene. After transformation, the direct repeats allow for the removal of the selection marker by a (second) homologous recombination event. The removal of the amdS marker can be done by plating on fluoro-acetamide media, resulting in the selection of marker-gene-free strains. Using this strategy of transformation and subsequent counterselection, which is also described as the “MARKER-GENE FREE” approach in EP 0 635 574, the amdS marker can be used indefinitely in strain modification programs. The general procedure for gene disruption is depicted in FIG. 1. The general design of deletion vectors was previously described in EP635574B and WO 98/46772 and the use of general cloning vector pGBDEL for constructing deletion vectors and the counterselection procedure were inter alia. described in WO06/040312.

### Strains

WT 1: This Aspergillus niger strain is used as a wild-type strain. This strain is deposited at the CBS Institute under the deposit number CBS 513.88.
WT 2: This A. niger strain is a WT 1 strain comprising a deletion of the gene encoding glucoamylase (glaA). WT 2 was constructed by using the “MARKER-GENE FREE” approach as described in EP 0 635 574 B1. In this patent it is extensively described how to delete glaA specific DNA sequences in the genome of CBS 513.88. The procedure resulted in a MARKER-GENE FREE glaA recombinant A. niger CBS 513.88 strain, possessing finally no foreign DNA sequences at all.

To disrupt the pepA gene encoding the major extracellular aspartic protease PepA in WT2, pepA specific DNA sequences in the genome of WT2 were deleted, as described by van den Hombergh et al. (van den Hombergh J P, Sollewijn Gelpke M D, van de Vondervoort P J, Buxton F P, Visser J. (1997)—Disruption of three acid proteases in Aspergillus niger—effects on protease spectrum, intracellular proteolysis, and degradation of target proteins—Eur J. Biochem. 247(2): 605-13). The procedure resulted in a MARKER-GENE FREE GBA 401 strain, with the pepA gene inactivated in the WT2 strain background.

To delete the oacA gene, annotated as An14g06860 in Pel et al, (2007 Genome sequencing and analysis of the versatile cell factory Aspergillus niger CBS 513.88. Nature Biotech 25:221-231) in GBA 401, the method as earlier described in detail in WO05/095624 was used to generate Aspergillus niger GBA 405 (ΔglaA, ΔpepA, ΔoacA).

To delete the amdR gene, annotated as An14g04000 in Pel et al, (2007 Genome sequencing and analysis of the versatile cell factory Aspergillus niger CBS 513.88. Nature Biotech 25:221-231) in GBA 405, encoding an acetamidase regulator in GBA 405, the method as earlier described in detail in WO05/095624 was used to generate Aspergillus niger GBA 406 (ΔglaA, ΔpepA, ΔoacA, ΔamdR).

Aspergillus tubingensis AN205 was kindly provided by C. M. O'Gorman and P.S. Dyer. It is cloned as described below by using primers 1 and 2 as described in table 3 on MAT1-2 containing isolate Aspergillus tubingensis AN205. The A. tubingensis MAT1-2 locus is analogous to other MAT1-2 loci in Aspergilli as indicated in Table 2.

TABLE 2
Alignment of MAT-locus proteins from different Aspergillus species
(A. tubingensis, A. parasiticus, A. flavus, A. fumigatus,
Neosartorya fischeri, A. nidulans respectively).

| | | |
|---|---|---|
| Mat1-2 | A. tubingensis (Atu) | MTSIPQALKPTTETSDKLTELLWQDALRHLG |
| Mat-2 | A. parasiticus (Apa) | MTTVPIAMKTTAESTDKLTELLWQDALRHLE |
| Mat-2 | A. flavus (Afl) | MTTIPIAMKTTAESTDKLTELLWQDALRHLE |
| Mat-2 | A. fumigatus (Afu) | MATVPIAMKPAAESTDTLTELLWQDALRHLE |
| Mat-2 | N. fischeri (Nfi) | MATIPITMKSAVDSTDTFTELLWQDALRHLE |
| Mat-2 | A. nidulans (And) | MAAVSIAMKSPTQSPDSITELLWKDALRHLG |

| | |
|---|---|
| Atu | STNNEVLLPINVIDMIGQTNVNKIKSRLGALIGASVVAFADESINAVRVMRTPAFSGTAI |
| Apa | STNNEVLLPINVTDMIGQSNVDKIRTRLGALIGAPVVAFVDETINALRVMRTPAFSGSVV |
| Afl | STNNEVLLPINVTDMIGQSNVDKIRTRLGALIGAPVVAFVDETINALRVMRTPAFSGSVV |
| Afu | STNNEVLLPINVTDMIGQDNVDKIKTRLGALIGAPVVAFVDETIKALRVMRTPAFSGTAV |
| Nfi | SMNNEVLLPINVTDMIGQDNVDKIKTH-SALIGAPVVAFVDKSIEALRVMRTPAFSGRAI |
| And | STNDEVLLPTNVVDIIGQDNVEKIKSRLSALLGAPVVSFVDESINALRVLRTPTFSGSSI |
| Atu | SVASH----------------------------E-------------------------- |
| Apa | SVASH----------------------------DRISNLEKEITEASGRTHGKSALPTKS |
| Afl | SVASH----------------------------DRISNLEKEITEASGRTHGKSALPTKS |
| Afu | SVASHGEAVKTNKVTVTESFAPRGKPVGPLKAPK-------------------------- |
| Nfi | SVASHGAALNADKVAATESFKPRGKPAGPMKAPK-------------------------- |
| And | SVASPSRALDSWPSEPPNKPRPASMKPA-----K-------------------------- |
| Atu | --VPRPPNAFILYRQHHHPKLKEAHPNLSNNEISVILGKQWKAESEDIRVEFRALADELK |
| Apa | TKVPRPPNAFILYRQHHHPRIKEAYPDFTNNEISIILGKQWKAESEEVKMQFRNMAEELK |
| Afl | K-VPRPPNAFILYRQHHHPRIKEAYPDFTNNEISIILGKQWKAESEEVKMQFRNMAEKLK |
| Afu | --VPRPPNAFILYRQHHHPKIKEAYPDYSNNDISVMLGKQWKDENEEIKTQFRNLAEELK |
| Nfi | --VPRPPNAFILYRQRHHPKIKEEYPDFSNNDI--MLGKQWKDEPEEVKAQFRNLAEELK |
| And | --IPRPPNAFILYRQHHYPKVKEARPDLSNNEISVIIGKKWRAEPEEGKLHFKNLAEEFK |
| Atu | RKHAEAHPDYQYTPRKPSEKKRRTSSR---RNSKQT----VENKSPDLTTASTIST---S |
| Apa | KKHAEDHPDYHYTPRKPSEKKRRASSR---QYSKPT----KRQKSPALTNDTSDSS---T |
| Afl | KKHAEDHPDYHYTPRKPSEKKRRASSR---QYSKPT----KRQKSPALTNDTSDSS---T |
| Afu | KKHAEDHPDYHYTPRKPSERKRRTSSR---QFSKNT----KPAALRDTPASMNISSDVST |
| Nfi | KKHAEDHPNYYYTPRKPSERKRRASSR---QFSKNTKSAAVLDIPASMNVASDVST---- |
| And | KKHAEEYPDYQYTPRKPSEKKRRAASRISPKNSKRT----VALENPGSMTA--------- |
| Atu | PSMDTYGPINDMGIEGTMNIFTEYNMPEIYPSDLICSDIMPSELPLLAADHFQLDTETLG |
| Apa | PSMYSGMQLDNIPVDASLD-----NLADI---DIVLS---PDELPRDCG--LQFDSVAFD |
| Afl | PSMYSGMQLDNIPVDASLD-----NLADI---DIVLS---PDELPRDCG--LQFDSVAFD |
| Afu | PAMLEGMPVGEIDFNAAFE-----DVPGI-------NAIMTSN-SILKNQQYHFEPNAF- |
| Nfi | PAMHQGMPVGEIDFNAAFE-----GVPDM---DVIMS---PTGIPEDQQFHF----EPNG |
| And | PSSNVFTPQMYPGIQNGQLAGAGY----IGYLDGLNSMVNTGG---LTDEPTNFGTNAFN |
| Atu | -SLIGQVHTDIGKGTGVYASPSAHVNLTDRHLGETVNFSEFISDLY |
| Apa | -NFLQQVQGDCGK---TAATLFPQFNFTERPVGESFEFSDLIADCY |
| Afl | -NFLQQVQGDCGK---TAATLFPQFNFTERPVGESFEFSDLIADCY |
| Afu | -DLMNQVQNDYNK-TALY----QQLSLPEGQIGENFEFTDFISDCF |
| Nfi | FDLIHQMENDYNK-AALYQQPS----LAEGPVGENYEFSDFITDCF |
| And | -SLFQQPQSDYGR-TALF----PQLEFAGPSLGDSLEFPEFAADYF |

TABLE 3
Primers used for PCR amplification of DNA
fragments of A. tubingensis AN205 WT1-MAT1-2

| No | PCR primer sequence | linker |
|---|---|---|
| 1 | cacgcgttacgctgagggaacatgg | Mlu1 |
| 2 | cacgcgtaggctatcacgagacctcac | Mlu1 |
| 3 | agaattccggtcctcgcaaatg | EcoR1 |
| 4 | agaattcggtggttggtcagacaag | EcoR1 |

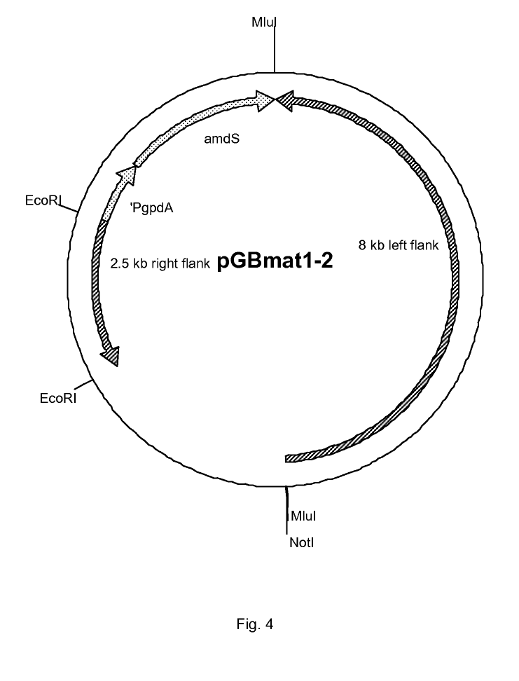

Primers used for PCR fragments are generated with primer pairs 1+2 and 3+4 as indicated in table 3. They are cloned into the vector pGBDeI resulting in plasmid pGBMAT1-2 as explained in FIG. 4. This construct is transformed to A. niger strain CBS513.88 using the amdS gene as a selection marker. This results in an A. niger transformant containing the MAT1-2 locus instead of the MAT1-1 locus, designated WT1-MAT1-2.

Sexual Crosses

Sexual crosses of the A. niger strains are set up using conventional techniques. They are set-up on agar plates. Several agar plates containing suitable types of medium are used: corn-meal agar, V-8 juice agar, malt extract agar, alphacell agar, oatmeal agar and soil extract agar as described in Kwon-Chung K. J. and Sugui J. A. 2009 (Sexual reproduction in Aspergillus species of medical or economical importance. Trends in Microbiology 17: 481-487). Also a minimal medium as described by Witteveen et al (1985) is used. The media are supplemented with 0.1% to 5% glucose and 1 to 20 g/l CaCO₃ or Na₂CO₃ or K₂CO₃. Crossing is executed so that strains GBA 406 and WT1-MAT1-2 are inoculated on the agar plates at a distance from each other, at 5 cm distance on the agar plates. The plates may than taped shut and placed in a container with increased pCO₂. After incubation during 1 to 12 months in the dark, the plates are examined for sexual structure formation. The sexual structures are taken from the plates and washed in sterile water. After this they are cut open with a scalpel and ascospores thus released from the sexual structures are diluted and plated on Potato Dextrose Agar (PDA). Of the colonies appearing, 20 were examined for properties differing from the two parental strains. Their genotypes are assessed using a PCR approach. Recombination of the mating type genes and mutations as listed in table 4 are examined.
In table 4 the genotype of the 20 recombinants is given. Recombination of the properties illustrates the effectiveness of the sexual cross.

TABLE 4
Genotype of parents and offspring of crossing in example I

| | | | | | found in 20 |
|---|---|---|---|---|---|
| genotypes | mating type | ΔAn14g06860 | Δan14g04710 | ΔAn14g04000 | progeny |
| WT1-MAT1-2 | MAT1-2 | Present | Present | Present | 2 |
| GBA 406 | MAT1-1 | Absent | Absent | Absent | 2 |
| combination 1 | MAT1-2 | Present | Absent | Absent | 1 |
| combination 2 | MAT1-1 | Absent | Absent | Absent | 0 |
| combination 3 | MAT1-1 | Present | Absent | Absent | 2 |
| combination 4 | MAT1-2 | Absent | Present | Absent | 1 |
| combination 5 | MAT1-2 | Present | Present | Absent | 2 |
| combination 6 | MAT1-1 | Absent | Present | Absent | 1 |
| combination 7 | MAT1-1 | Present | Present | Absent | 0 |
| combination 8 | MAT1-2 | Absent | Absent | Present | 2 |
| combination 9 | MAT1-2 | Present | Absent | Present | 1 |
| combination 10 | MAT1-1 | Absent | Absent | Present | 2 |
| combination 11 | MAT1-1 | Present | Absent | Present | 1 |
| combination 12 | MAT1-2 | Absent | Present | Present | 2 |
| combination 13 | MAT1-2 | Present | Present | Present | 0 |
| combination 14 | MAT1-1 | Absent | Present | Present | 1 |

Genotypes found in 20 isolates from a sexual cross of WT1-MAT1-2 with GBA 406.

1. A process for preparing filamentous fungal strain having a sexual cycle, comprising subjecting an acceptor filamentous fungal strain, having none or not more than one type MAT locus, to recombination in which at least one mating type locus gene and optionally sex related gene, from another species from the same genus as the acceptor filamentous fungal strain, is introduced, into the acceptor filamentous fungal strain to produce a filamentous fungal strain having a sexual cycle.

2. The process according to claim 1, wherein said at least one mating type locus gene and optionally sex related gene originates from at least one donor filamentous fungus.

3. The process according to claim 1, wherein said acceptor filamentous fungal strain is Aspergillus niger, Aspergillus niger var. awamori, Aspergillus terreus, Aspergillus aculeatus, Aspergillus candidus, Aspergillus japonicus, Aspergillus sojae, Aspergillus vadensis, Aspergillus brasiliensis, Aspergillus carbonarius, Aspergillus lacticoffeatus, Aspergillus aureus, Aspergillus oryzae or Chrysosporium lucknowense.

4. The process according to claim 3, wherein said acceptor filamentous fungal strain is Aspergillus niger.

5. The process according to claim 1, wherein said acceptor filamentous fungal strain has a MAT 1-1 locus, and in which a MAT1-2 locus is introduced.

6. The process according to claim 5, wherein MAT1-2 locus replaces the MAT1-1 locus.

7. The process according to claim 2, wherein said donor filamentous fungal strain is of the genus, Acremonium, Penicillium, Emericella, Trichoderma, Hypocrea or Aspergillus.

8. The process according to claim 2, wherein said donor filamentous fungal strain is of the genus Aspergillus, but does not comprise Aspergillus niger.

9. The process according to claim 1, wherein said acceptor filamentous fungal strain is of the genus Aspergillus and said mating type locus is derived from a donor filamentous fungal strain belonging to the genus Aspergillus.

10. The process according to claim 2, wherein said donor filamentous fungal strain is Aspergillus tubingensis.

11. The process according to claim 10, wherein said donor filamentous fungal strain is Aspergillus tubingensis AN205.

12. The process according to claim 10, wherein said acceptor filamentous fungal strain is subjected to recombination with an isolated polynucleotide which comprises at least one of:
(a) the nucleotide sequence set out in SEQ ID NO: 1; or
(b) a nucleotide sequence having at least 70% sequence identity with the nucleotide sequence of SEQ ID NO: 1; or
(c) a sequence which is degenerate as a result of genetic code to a sequence as defined in any one of (a), (b) or (d); or
(d) a nucleotide sequence which is a reverse complement of a nucleotide sequence as defined in (a), (b), or (c), or
(e) a nucleotide sequence encoding a polypeptide comprising an amino acid sequence set out in SEQ ID NO: 3 or a polypeptide having at least 70% sequence identity with sequence SEQ ID NO: 3, or
(f) a nucleotide sequence encoding a polypeptide comprising an amino acid sequence set out in SEQ ID NO: 4 or a polypeptide having at least 70% sequence identity with sequence SEQ ID NO: 4.

13. The process according to claim 1, wherein said sex-related gene comprises a gene involved in mating behaviour, fertilization, spermatogenesis, and/or sex determination.

14. A filamentous fungal strain obtainable by the process of claim 1.

15. The filamentous fungal strain according to claim 14, wherein said strain is of species Aspergillus niger or Aspergillus oryzae.

16. The filamentous fungal strain according to claim 14, having a heterothallic sexual cycle.

17. An isolated polynucleotide which comprises at least one of:
(a) the nucleotide sequence set out in SEQ ID NO: 1; or
(b) a nucleotide sequence having at least 70% sequence identity with the nucleotide sequence of SEQ ID NO: 1; or
(c) a sequence which is degenerate as a result of genetic code to a sequence as defined in any one of (a), (b) or (d); or
(d) a nucleotide sequence which is a reverse complement of a nucleotide sequence as defined in (a), (b), or (c).

18. An isolated polypeptide comprising an amino acid sequence set out in SEQ ID NO: 3 or a polypeptide encoded by a nucleotide sequence of claim 16 or a variant polypeptide thereof having at least 80% sequence identity with sequence SEQ ID NO: 3.

19. An isolated polypeptide comprising an amino acid sequence set out in SEQ ID NO: 4 or a polypeptide encoded by a nucleotide sequence of claim 16 or a variant polypeptide thereof having at least 80% sequence identity with sequence SEQ ID NO: 4.

20. A vector comprising the polynucleotide of claim 17.

21. A filamentous fungal strain comprising a polynucleotide of claim 17.

22. A process for producing filamentous fungal strain progeny, comprising sexually crossing individuals with two opposite mating types of said filamentous fungal strain according to claim 14, and isolating progeny.

23. The process according to claim 22, wherein said process is without the use of a marker.

24. The process according to claim 23, wherein two opposite mating type filamentous fungal strains from a pool of strains, that are of vertical or horizontal lineage, are sexually crossed.

25. A process for selecting a filamentous fungal strain with desired phenotype comprising screening a library of progeny produced according to claim 22, and screening and/or selecting at least one strain with a desired phenotype.

26. The process according to claim 22, wherein two strains of different sex are plated on agar plates at a distance from each other.

27. The process according to claim 26, wherein said distance is at least 1 cm, optionally at least 3 cm.

28. A filamentous fungal strain having a heterothallic sexual cycle obtainable by said process of claim 22.

29. A filamentous fungal strain obtainable by the process of claim 22, with MAT locus removed and/or recombined to acceptor fungus species type of MAT locus.

30. A filamentous fungal strain obtainable by the process of claim 22, with additional gene of interest introduced for production of a product of interest, optionally comprising an enzyme, protein or metabolite.

31. A process for preparing at least one compound of interest comprising:
a. cultivating said filamentous fungal strain according to claim 30, under conditions conducive to producing said compound; and
b. recovering said compound of interest from a cultivation medium.

32. The process according to claim 31, wherein said compound of interest is a polypeptide.


SEQUENCE LISTING (9 sequences)

SEQ ID NO: 1 - DNA, 1110 bp, Artificial Sequence
  Aspergillus tubingensis
atgacttctatacctcaagcattgaagccaactactgagaccagcgacaagcttacagagctactttggcaggatgccttgcgccatcttggatccacgaataatgaagttctacttccaataaatgtcatcgacatgatcggccagaccaatgttaacaagatcaagtctcgccttgggtaagacagctgtaaccacaattgatcaattatgtgctcatatatggttttagtgctctcataggtgcttcagtcgtggctttcgccgatgagtcaatcaacgctgttcgtgtcatgcgtactccagcgttttcaggcacagcaatatctgtagcatcccatgaggtgtcatgcgaatcaatcacagctcatatgaagaagcatgagcggccttctgttactgttaaggctgctaaggttcctcgtccgccaaatgcatttatcctgtaccgccaacatcatcatcctaagctcaaggaggctcacccgaatctttcaaataacgagatttgtgagtttccactactgtgcatacgttggataatcgactaatttccccagctgttattctagggaagcagtggaaggcagaatccgaggatatcagggtcgagtttagagccctggccgatgagttgaagcggaagcatgcagaagctcatcccgactaccaatacacaccacgtaaaccatcggaaaagaagcgtcgaacatcatctcgccgaaattccaaacagactgtagagaacaagtctccagatctcacgacggccagcaccatatcaacaagtccgtcaatggacacatatgggccgataaacgatatgggaattgaagggacgatgaatatctttacagaatacaatatgccagagatttaccccagcgacttgatatgcagcgacataatgccaagcgaactaccactgttagcggccgatcacttccagttagacacggagacattgggttcgttgatcgggcaggttcatacagacatcggaaaaggaacaggagtgtatgcctctccatcggcccatgtcaacttaacagaccgacatcttggcgaaactgtcaacttctcagaatttatttccgatctttattgaaa

SEQ ID NO: 2 - protein, 310 aa, Aspergillus tubingensis
MTSIPQALKPTTETSDKLTELLWQDALRHLGSTNNEVLLPINVIDMIGQTNVNKIKSRLGALIGASVVAFADESINAVRVMRTPAFSGTAISVASHEVPRPPNAFILYRQHHHPKLKEAHPNLSNNEISVILGKQWKAESEDIRVEFRALADELKRKHAEAHPDYQYTPRKPSEKKRRTSSRRNSKQTVENKSPDLTTASTISTSPSMDTYGPINDMGIEGTMNIFTEYNMPEIYPSDLICSDIMPSELPLLAADHFQLDTETLGSLIGQVHTDIGKGTGVYASPSAHVNLTDRHLGETVNFSEFISDLY

SEQ ID NO: 3 - protein, 92 aa, Aspergillus tubingensis
EVPRPPNAFILYRQHHHPKLKEAHPNLSNNEISVILGKQWKAESEDIRVEFRALADELKRKHAEAHPDYQYTPRKPSEKKRRTSSRRNSKQT

SEQ ID NO: 4 - protein, 96 aa, Aspergillus tubingensis
MTSIPQALKPTTETSDKLTELLWQDALRHLGSTNNEVLLPINVIDMIGQTNVNKIKSRLGALIGASVVAFADESINAVRVMRTPAFSGTAISVASH

SEQ ID NO: 5 - protein, 350 aa, Aspergillus niger
MPPQQLEELLRYLQDTRAKANNQVAHENEQSTARSKPPLDTNNESAVQGSTNQRSHQTRGKRPQDGKRRPLNSFIAFRSYYSVMFPDLTQKAKSGILRFLWQNDPFKAKWAIVAKAYSIIRDDHDNGVSLETFLKLNTNLIGIIEPDRYLDIMGWELNVDGQQQYTMAKVKITATPEAELSTNYSVDDIVKNCYDTGYVSRDKCKKKTDHNKNAPVMAFAAQPTLIIHENNSIQINGNNTVVTGDCDGNAAVESTFPQNFSPSPGDMGTMLAEASLGESEVLHGRGYQLYERGPIVANEYEIDDGMLNLWGENPAMLPSYVSAGQCTLPPHDPCVPDPMANINIDHYLNL

SEQ ID NO: 6 - DNA, 25 bp, Artificial Sequence
  PCR primer
cacgcgttacgctgagggaacatgg

SEQ ID NO: 7 - DNA, 27 bp, Artificial Sequence
  PCR Primer
cacgcgtaggctatcacgagacctcac

SEQ ID NO: 8 - DNA, 22 bp, Artificial Sequence
  PCR primer
agaattccggtcctcgcaaatg

SEQ ID NO: 9 - DNA, 25 bp, Artificial Sequence
  PCR primer
agaattcggtggttggtcagacaag